SEQUENCE LISTING (3 sequences)

SEQ ID NO: 1 - protein, 525 aa, Saccharomyces bayanus
MNTYSEKTSLVQDECLAKMIQNGHSRRMGSVEDLYAALNRQKLYRNFSTYSELNDYCTKDQLALALRNICLKNPTLLHIVLPARWPDHENYYLSSEYYSQPHPKHDYISVLPELKFDGVILNEQPEHNALMKQILEELKDSNGSYTAKIFKLTTALTIPYAGPTSPTWRLICLPEEGYTDKWKKFIFLSNHCMCDGRTSIHFFQDLRDELNNIKTPPKKLDYIFQYEKDYQLLRKLPEPIENMIDFRPPYMFIPKSLISGFIYSHLRFSSKGVCTRMDELEKNDDIVTEIITISPSELQKIRTKIKSNIPGKCTITPFLEVCWFVSLHKWGKFFKPLKFEWLTDVFIPADCRSLLPEDEDVRAMYRYGANVGFVDFTPWISEFNMNDSKENFWPLIAHYHEVISGAINDKKHLNGLGFNIQGLVQKYVNIDKVMRDRALGKSRGGTLLSNVGIFHQSEETDSRYSIRDLAFGQFQGSWHQAFSLGVCSTNVKGMNIVISSTKNAVGSQELLEELCAMYKALLLDP

SEQ ID NO: 2 - DNA, 1578 bp, Saccharomyces bayanus
  source 1..1578: mol_type = genomic DNA
atgaacacctactctgagaagacctctcttgttcaggacgagtgtctggctaagatgattcagaacggtcactctcgacgaatgggctctgtcgaggacctttacgccgccctcaaccgacagaagctctaccgaaacttctctacttactctgagctgaacgattactgcactaaggatcagctcgctcttgctctccgaaacatttgtctgaagaaccccactctccttcacattgttcttcccgctcgatggcccgatcacgagaactactacctttcttctgagtactactctcagccccaccccaagcacgattacatctctgttcttcccgagctgaagttcgatggtgtgattctcaacgagcagcccgagcacaacgcccttatgaagcagattcttgaggagcttaaggattccaacggttcttacactgctaagattttcaagctcactaccgctctcactattccctacgctggtcccacttctcccacttggcgactgatttgtctgcccgaggagggatacaccgataagtggaagaagtttattttcctttccaaccactgcatgtgtgatggtcgaacctctattcacttctttcaggatctccgagatgagcttaacaacatcaagactccccccaagaagctcgactacattttccagtacgagaaggactaccagcttctccgaaagctccccgagcccattgagaacatgattgattttcgacccccctacatgtttattcccaagtcccttatttccggcttcatttactcccaccttcgattctcctctaagggtgtgtgtacccgaatggacgagcttgagaagaacgacgatattgttactgagatcatcaccatctctccctctgagcttcagaagattcgaactaagatcaagtctaacattcccggcaagtgcaccatcactcccttccttgaggtttgttggtttgtttctctccacaagtggggcaagtttttcaagcccctcaagttcgagtggcttaccgatgtttttattcccgctgattgccgatctctgctccccgaggacgaggacgtgcgagctatgtaccgatacggcgctaacgtcggttttgttgacttcactccctggatttctgagtttaacatgaacgactctaaggagaacttctggccccttattgctcactaccacgaggttatttctggtgccatcaacgacaagaagcacctcaacggtcttggtttcaacattcagggccttgtccagaagtacgtcaacattgacaaggtgatgcgagatcgagcccttggtaagtcccgaggaggcaccctgctctctaacgtgggtattttccaccagtctgaggagactgactcccgatactctatccgagatctcgctttcggtcagtttcagggttcttggcaccaggctttctctctcggtgtttgttccactaacgtgaagggaatgaacattgttatttcttccactaagaacgccgtgggttcccaggagctccttgaggagctttgtgccatgtacaaggctctgctccttgacccctaa

SEQ ID NO: 3 - DNA, 13904 bp, synthetic construct
  source 1..13904: mol_type = other DNA
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgaacacctactctgagaagacctctcttgttcaggacgagtgtctggctaagatgattcagaacggtcactctcgacgaatgggctctgtcgaggacctttacgccgccctcaaccgacagaagctctaccgaaacttctctacttactctgagctgaacgattactgcactaaggatcagctcgctcttgctctccgaaacatttgtctgaagaaccccactctccttcacattgttcttcccgctcgatggcccgatcacgagaactactacctttcttctgagtactactctcagccccaccccaagcacgattacatctctgttcttcccgagctgaagttcgatggtgtgattctcaacgagcagcccgagcacaacgcccttatgaagcagattcttgaggagcttaaggattccaacggttcttacactgctaagattttcaagctcactaccgctctcactattccctacgctggtcccacttctcccacttggcgactgatttgtctgcccgaggagggatacaccgataagtggaagaagtttattttcctttccaaccactgcatgtgtgatggtcgaacctctattcacttctttcaggatctccgagatgagcttaacaacatcaagactccccccaagaagctcgactacattttccagtacgagaaggactaccagcttctccgaaagctccccgagcccattgagaacatgattgattttcgacccccctacatgtttattcccaagtcccttatttccggcttcatttactcccaccttcgattctcctctaagggtgtgtgtacccgaatggacgagcttgagaagaacgacgatattgttactgagatcatcaccatctctccctctgagcttcagaagattcgaactaagatcaagtctaacattcccggcaagtgcaccatcactcccttccttgaggtttgttggtttgtttctctccacaagtggggcaagtttttcaagcccctcaagttcgagtggcttaccgatgtttttattcccgctgattgccgatctctgctccccgaggacgaggacgtgcgagctatgtaccgatacggcgctaacgtcggttttgttgacttcactccctggatttctgagtttaacatgaacgactctaaggagaacttctggccccttattgctcactaccacgaggttatttctggtgccatcaacgacaagaagcacctcaacggtcttggtttcaacattcagggccttgtccagaagtacgtcaacattgacaaggtgatgcgagatcgagcccttggtaagtcccgaggaggcaccctgctctctaacgtgggtattttccaccagtctgaggagactgactcccgatactctatccgagatctcgctttcggtcagtttcagggttcttggcaccaggctttctctctcggtgtttgttccactaacgtgaagggaatgaacattgttatttcttccactaagaacgccgtgggttcccaggagctccttgaggagctttgtgccatgtacaaggctctgctccttgacccctaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

(19) United States
(12) Patent Application Publication
(10) Pub. No.: US 2026/0234575 A1
(43) Pub. Date: Aug. 13, 2026

(54) NOVEL MUTANTS OF ACETYL-TRANSFERASE SB-ATF

(71) Applicant: DSM IP ASSETS B.V., Maastricht (NL)

(72) Inventors: Valmik Kanubhai VYAS, Winchester, MA (US); René Marcel DE JONG, Amsterdam (NL); Anna SYMBOR-NAGRABSKA, Reading, MA (US); Peter Louis HOUSTON, Boston, MA (US)

(21) Appl. No.: 19/149,990

(22) PCT Filed: Jan. 26, 2024
(86) PCT No.: PCT/EP2024/051845

§ 371 Date: Jul. 22, 2025

Related U.S. Application Data
(60) Provisional application No. 63/482,157, filed on Jan. 30, 2023.

(30) Foreign Application Priority Data
May 25, 2023 (EP) 23175418.5

(51) Int. Cl. C12N 9/10; C12P 7/6436

(52) U.S. Cl. CPC C12N 9/1029; C12P 7/6436; C12Y 203/01084

(57) ABSTRACT

The present invention is related to a process for transformation of low activity enzymes involved in acetylation of retinoids into high activity enzymes, particular high activity towards acetylation of retinol into retinyl acetate.

The present invention is related to a process for transformation of low activity enzymes involved in acetylation of retinoids into high activity enzymes, particular high activity towards acetylation of retinol into retinyl acetate.

Retinyl acetate is an important intermediate or precursor for production of retinoids, particularly such as vitamin A. Retinoids, including vitamin A, are one of very important and indispensable nutrient factors for both humans and animals which must be supplied via diet. Retinoids promote well-being, inter alia in respect of vision, the immune system and growth.

Current chemical production methods for retinoids, particularly vitamin A and precursors thereof, have some undesirable characteristics such as e.g. high-energy consumption, complicated purification steps and/or undesirable by-products. Therefore, over the past decades, other approaches to manufacture retinoids, particularly vitamin A and precursors or intermediates thereof, have been investigated, including microbial conversion steps, which would be more economical as well as ecological.

In general, the biological systems that produce retinoids are industrially intractable and/or produce the compounds at such low levels that its isolation on industrial scale is not practicable of economic interest. There are several reasons for this, most of all instability issues of the intermediates/retinoids and/or the relatively high accumulation of by-products.

One way of dealing with instability issues would be production of acetylated product forms. Acetylation of carotenoids, such as e.g. astaxanthin or zeaxanthin, such as by action of Atf1 from Saccharomyces bayanus (SbATF1), has been previously reported (WO2014096992), with acetylation of for instance zeaxanthin in the range of up to 90 wt %. However, when using said enzymes in the acetylation of retinoids, particularly acetylation of retinol, the percentage of acetylation is only in the range of about max. 10% based on total retinoids (see e.g. WO2019058001), which is far too low to establish a sustainable and economically feasible industrial process for bio-production of vitamin A.

Thus, there is a strong need towards improvement of enzymes to be used in acetylation of retinoids.

Surprisingly, we now could identify amino acid positions in fungal acetyl transferase enzymes (ATFs), such as e.g. originated from Saccharomyces bayanus and other yeast, which are critical for formation of acetylated retinoids, particularly conversion of retinol into retinyl acetate. Modification of certain amino acids results in increased formation of retinyl acetate as compared to the respective wild-type enzymes, such as e.g. an increase of at least about 10% when compared to acetylation of retinol using the respective non-modified enzymes, e.g. wt-ATF1 from Saccharomyces bayanus as shown in SEQ ID NO:1.

Thus, in one embodiment, the present invention is directed to a method for modification of enzymes involved in/catalyzing the acetylation of retinoids, more particularly fungal ATFs, comprising the introduction of at least one modification, e.g. amino acid substitution, such as e.g. comprising 1 to 9, such as e.g. at least 2, amino acid substitution(s), on position(s) specified herein. Upon introduction of the specified amino acid substitution(s), said non-modified ATF, particularly fungal ATF, such as e.g. ATF1 originated from Saccharomyces, e.g. ATF1 from Saccharomyces bayanus (SbATF) according to SEQ ID NO:1, is modified or transformed into a modified or mutated ATF with increased activity, wherein upon expression of such modified enzyme in a suitable expression system, including a suitable host cell, as specified herein, the percentage of retinyl acetate obtained from acetylation of retinol could be increased by at least about 10%, such as increased by more than 700% as compared to the percentage of retinyl acetate using the respective non-modified or wild-type enzyme.

Particularly, the present invention is directed to a method for modification of an enzyme catalyzing the acetylation of retinoids, particularly retinol into retinyl acetate, preferably a fungal enzyme, comprising introduction of at least 1 amino acid substitution at one or more position selected from the group consisting of amino acid residue 34, 35, 346, 371, 373, 419, 434, 437, 478, and combinations thereof in the polypeptide according to SEQ ID NO:1, wherein upon introduction of the one or more amino acid substitution(s) the activity towards formation of retinyl acetate is increased by at least about 10%, such as in the range of 10 to 760% compared to the activity using the respective non-modified enzyme including an enzyme according to SEQ ID NO:1, preferably wherein the at least one modification, e.g. amino acid substitution, such as e.g. comprising 1 to 9 amino acid substitution(s), on position(s) specified herein, preferably amino acid residues corresponding to 34, 35, 346, 371, 373, 419, 434, 437, 478, and/or combinations thereof in the polypeptide according to SEQ ID NO:1 is/are different from L34, Y35, F346, V371, F373, N419, M434, R437, W478 and combinations thereof, i.e. corresponding to the respective amino acid residues in SEQ ID NO:1.

More particularly, the present invention is directed to a method for modification of an enzyme catalyzing the acetylation of retinoids, particularly retinol into retinyl acetate, preferably a fungal enzyme, comprising introduction of at least 2 amino acid substitutions at position selected from amino acid residue 34 and 434 in the polypeptide according to SEQ ID NO:1, wherein upon introduction of said at least two amino acid substitutions the activity towards formation of retinyl acetate is increased by at least about 10%, e.g. at least about 25, 20, 25, 30, 35, 40, 45, 50, 55, 60, 65, 70, 75, 80, 85, 90, 95, 100, 120, 150, 180, 200, 230, 250, 280, 300, 330, 350, 380, 400, 430, 450, 480, 500, 550, 600, 650, 700, 750, 800, such as in the range of 10 to 760% compared to the activity using the respective non-modified enzyme including an enzyme according to SEQ ID NO:1, preferably wherein the amino acid residues on positions corresponding to amino acid residue 34 and 434 in SEQ ID NO:1 are different from L34 and M434, more preferably wherein the amino acid substitutions on position corresponding to L34 and M434 in the polypeptide according to SEQ ID NO:1 are selected from L34F and M434V.

As used herein, a non-modified retinol acetylating enzyme as defined herein having “low activity” towards acetylation of retinol means a percentage of retinyl acetate in the range of about 10 wt % or lower based on total retinoids produced in a suitable retinoid-producing host cell expressing said non-modified enzyme. A modified enzyme as defined herein having “increased activity” towards acetylation of retinol means a percentage of retinyl acetate of about 11 wt % to 86 wt % based on total retinoids produced in a suitable retinoid-producing host cell expressing said modified enzyme, i.e. said modified enzyme being generated via introduction of one or more amino acid substitution(s) as defined herein transforming an enzyme with low (enzyme or acetylating) activity into and enzyme with increased (enzyme or acetylating) activity as defined herein. As shown, the activity towards acetylation of retinol could be increased by at least about 10%, such as e.g. increased by 20, 30, 40, 50, 60, 70, 80, 90, 100, 150, 200, 250, 300, 350, 400, 450, 500, 550, 600, 650, 700, 750, or more.

More particularly, the present invention is directed to a process for production of retinol acetylating enzymes having increased activity towards acetylation of retinol into retinyl acetate, said process comprising:
(a) providing a fungal enzyme involved in acetylation of retinol, particularly ATF originated from Saccharomyces, preferably SbATF, wherein said non-modified enzyme expressed in a suitable host cell and under suitable culture conditions is capable of producing retinyl acetate from retinol based on total retinoids in the range of 10 wt % or less;
(b) introduction of 1 or more amino acid substitution(s), such as at least 1 to 9, preferably at least 2, amino acid substitution(s) into the enzyme of (a) to generate a modified enzyme involved in acetylation of retinol, wherein the ability to convert retinol into retinyl acetate is increased by at least about 10%, such as e.g. by at least 10 to 760% when compared to the acetylation activity of the respective non-modified enzyme of step (a);
wherein the 1 or more, such as e.g. at least 2, amino acid substitution(s) are being located at position(s) corresponding to amino acid residue(s) being selected from the group consisting of position 34, 35, 346, 371, 373, 419, 434, 437, 478 and combinations thereof in SEQ ID NO:1 and wherein the substitute amino acid(s) is/are different from L34, Y35, F346, V371, F373, N419, M434, R437, W478 and/or combinations thereof, preferably wherein the at least 2 amino acid substitutions are at positions corresponding to position 34 and 434 in the polypeptide according to SEQ ID NO:1.

In one embodiment, the present invention is related to a modified enzyme involved in acetylation of retinol into retinyl acetate, particularly fungal enzyme comprising one or more modification(s), such as at least 1, 2, 3, 4, 5, 6, 7, 8, 9, such as e.g. at least 2, amino acid substitution(s), said amino acid substitution(s) being located at position(s) corresponding to amino acid residue(s) being selected from the group consisting of position 34, 35, 346, 371, 373, 419, 434, 437, 478 and combinations thereof in the polypeptide according to SEQ ID NO:1, and wherein the substitute amino acid residue(s) are different from amino acids corresponding to L34, Y35, F346, V371, F373, N419, M434, R437, W478 and combinations thereof in the enzyme according to SEQ ID NO:1, preferably wherein the amino acid residue(s) to be substituted are corresponding to residues selected from the group consisting of L34, Y35, F346, V371, F373, N419, M434, R437, W478 and combinations thereof in a polypeptide according to SEQ ID NO: 1, more preferably wherein the substitute amino acid residue to be introduced at position corresponding to position 34, 35, or 478 in SEQ ID NO:1 is preferably phenylalanine, wherein the substitute amino acid residue to be introduced at position corresponding to position 346 or 419 in SEQ ID NO:1 is preferably leucine, wherein the substitute amino acid residue to be introduced at position corresponding to position 371 in SEQ ID NO:1 is preferably isoleucine, wherein the substitute amino acid residue to be introduced at position corresponding to position 373 in SEQ ID NO:1 is preferably alanine, and/or wherein the substitute amino acid residue to be introduced at position corresponding to position 434 or 437 in SEQ ID NO:1 is preferably valine.

Particularly, the modified enzyme according to the present invention and as defined herein is selected from a polypeptide with at least about 20%, such as 25, 30, 35, 40, 45, 50, 55, 60, 65, 70, 75, 80, 85, 90, 92, 95, 97, 98, 99% or up to 100% identity to SEQ ID NO:1, comprising one or more amino acid substitution(s) on position(s) selected from 34, 35, 346, 371, 373, 419, 434, 437 and/or 478 in SEQ ID NO: 1 being different from L34, Y35, F346, V371, F373, N419, M434, R437 or W478, preferably wherein the one or more amino acid substitution(s) are selected from the group consisting of L34F, Y35F, V371I, M434V, N419L, F346L, R437V, W478F, F373A, and combinations thereof.

The use of such modified enzyme in a process for production of retinoids, wherein said modified enzyme is expressed, particularly heterologous expressed, in a suitable host cell, particularly fungal host cell capable of retinol production, leads to an increase in retinyl acetate produced via acetylation of retinol by at least about 10% as compared to the process using the same conditions but the respective or corresponding wild-type enzyme, such as e.g. an ATF enzyme according to SEQ ID NO:1. Particularly, the use of such modified enzyme leads to titers of retinyl acetate in the range of up to 86 wt % based on total retinoids, as compared to the use of the respective non-modified enzyme with titers in the range of max. 10 wt % retinyl acetate based on total retinoids

The terms “acetyl transferase”, “retinol acetylating enzyme”, “enzyme having retinol acetylating activity”, “ATF” or “ATF1” are used interchangeably herein and refer to enzymes of EC class [EC 2.3.1.84] which are capable of catalyzing the conversion of retinol into retinyl acetate, including both naturally occurring enzymes and enzymes synthetically generated by the help of artificial intelligence. The terms “SbATF” and “SbATF1” are used interchangeably herein. An example of such an enzyme is shown in SEQ ID NO:1. As defined herein, ATFs capable of catalyzing the acetylation of retinol into retinyl acetate with a percentage of retinyl acetate based on total retinoids of about 10% or less are referred to herein as “non-modified” ATF or “wild-type” ATF, such as e.g. the SbATF according to SEQ ID NO.1.

A “modified” ATF as defined herein and particularly based on “non-modified” ATF, such as e.g. an enzyme with at least about 20% identity to SEQ ID NO:1 as defined herein, shows an increased activity, i.e. increase in the formation of retinyl acetate from conversion of retinol in the range of at least 10% based on total retinoids and as compared to retinyl acetate formation using the respective/corresponding wild-type enzyme, such as an enzyme according to SEQ ID NO:1.

In one embodiment, the modified ATF according to the present invention comprises at least 1, 2, 3, 4, 5, 6, 7, 8, 9, such as e.g. at least 2, amino acid substitution(s) as compared to the corresponding wild-type ATF, wherein one or more of the amino acid(s) corresponding to L34, Y35, F346, V371, F373, N419, M434, R437, and/or W478 in the polypeptide according to SEQ ID NO:1 is/are exchanged with a result that the enzymatic activity towards formation of retinyl acetate is increased by at least about 10% compared to the respective non-modified ATF, e.g. ATF according to SEQ ID NO:1, particularly with an increase in the percentage of retinyl acetate based on total retinoids from about 10 wt % to 86 wt % or even more.

The present invention includes a method for transforming/converting a low-activity enzyme (with respect to acetylation of retinol into retinyl acetate) also referred to herein as non-modified enzyme into a modified enzyme having increased activity towards acetylation of retinol into retinyl acetate as defined herein. An example of such non-modified enzyme is an enzyme with at least about 20% identity to SEQ ID NO:1 showing an acetylation activity of about 10%, i.e. when expressed in a suitable retinol producing host cell the percentage of retinyl acetate obtained from conversion/acetylation of retinol is in the range of 10 wt % or less based on total retinoids, particularly ATF1 isolated from Saccharomyces bayanus (SbATF1) including enzymes encoded by a polynucleotide according to SEQ ID NO:2.

Suitable non-modified enzymes including enzymes with at least about 20%, such as 25, 30, 35, 40, 45, 50, 55, 60, 65, 70, 75, 80, 85, 90, 92, 95, 97, 98, 99% or up to 100% identity to SEQ ID NO:1, including SbATF1 isolated/originated from Saccharomyces bayanus, are defined as enzymes showing an acetylation activity of about 10%, i.e. when expressed in a suitable retinol producing host cell the percentage of retinyl acetate obtained from conversion/acetylation of retinol is in the range of 10 wt % or less based on total retinoids, are obtainable from fungal enzymes, particularly enzymes obtainable from yeast, particularly enzymes comprising a highly conserved partial amino acid sequence of at least 7 amino acid residues selected from [NSDEHC]-H-x (3)-D-[GA] (motif in Prosite syntax, see: https://prosite.expasy.org/scanprosite/scanprosite_doc.html), preferably [N-H-x (3)-D-[GA], more preferably N-H-C-M-C-D-G, wherein “x” denotes an arbitrary amino acid and with the central histidine being part of the enzyme's binding pocket, corresponding to position N190 to G196 in the polypeptide according to SEQ ID NO:1.

Suitable non-modified enzymes can be selected from yeast, including but not limited to Saccharomyces, such as e.g. S. bayanus, S. cerevisiae, S. kudriavzevii, S. mikatae, S. pastorianus, S. arboricola, Candida, such as e.g. C. dubliniensis, C. albicans, C. intermedia, C. auris, C. lusitaniae, Wickerhamomyces, such as e.g. W. anomalus, Aspergillus, such as e.g. A. ellipticus.

Modified ATFs as defined herein are capable of converting retinol into retinyl acetate, particularly with conversion ratios being increased by at least about 10% as compared to conversion of retinol into retinyl acetate using the respective/corresponding non-modified enzyme, e.g. enzyme according to SEQ ID NO: 1, such as e.g. obtainable via expression of a modified ATF under suitable culture conditions including but not limited to cultivation on glucose, galactose, xylose with or without the combination of ethanol.

The enzymes as defined herein are used in the conversion of retinol into retinyl acetate, wherein the substrate (i.e. retinol) can be either cis-, trans- or a mix of cis-/trans-retinol in any possible ratio. Preferably, the retinol-mix to be used as substrate has high percentage of trans-retinol, such as e.g. at least about 65 to 98 wt % of trans-isomer based on total retinol in the host cell. Acetylation of said retinol-mix with at least about 65 to 98 wt % trans-retinol would lead to retinyl acetate with about the same ratio of trans to cis-retinyl acetate based on total retinyl acetate produced by the host cell.

In one specific embodiment, the present invention is related to conversion of retinol into retinyl acetate using a suitable host cell as defined herein comprising and expressing a modified enzyme as defined herein, wherein the retinol is a mix of trans- and cis-retinol and wherein the percentage of trans-retinol is in the range of at least about 65 to 98 wt % trans retinol based on total retinol.

The terms “conversion”, “enzymatic conversion”, “acetylation” or “enzymatic acetylation” in connection with enzymatic catalysis of retinol are used interchangeably herein and refer to the action of modified or non-modified ATF in the catalysis of retinol to retinyl acetate conversion resulting in a certain percentage of retinyl acetate based on total retinoids present/produced by a suitable host cell upon expression of said ATF, wherein an increase to 86 wt % or more retinyl acetate based on total retinoids can be achieved using a modified ATF as defined herein.

Suitable host cells according to the present invention include fungal host cells as well as microbial host cells such as e.g. E. coli. As used herein, the term “fungal host cell” particularly includes yeast cells, wherein the cell is a retinol-producing host cell, particularly a retinyl acetate-producing host cell, such as retinyl acetate-producing fungal host cell, including but not limited to Yarrowia or Saccharomyces, such as e.g. Yarrowia lipolytica or Saccharomyces cerevisiae.

The modified ATF enzyme might be used in an isolated form (e.g. in a cell-free system) or might be expressed in the suitable host cell, such as e.g. retinol-producing host cell, particularly fungal host cell as defined herein. Enzymes might be expressed as endogenous enzymes or as heterologous enzymes. Preferably, the modified enzymes as described herein are introduced and expressed as heterologous enzymes in a suitable host cell, such as e.g. a retinol-producing host cell, particularly fungal host cell as defined herein.

In one embodiment, the modified ATF enzyme as defined herein comprises an amino acid substitution at a position corresponding to residue 34 in the polypeptide according to SEQ ID NO:1 leading to substitution of the original residue by any amino acid except leucine, particularly through exchange of the original amino acid by phenylalanine, such as particularly via substitution of leucine by phenylalanine (e.g. L34F in SEQ ID NO:1). Said enzyme to be modified can be derived from an ATF with at least about 20% identity to SbATF shown in SEQ ID NO:1. Preferably, such modified enzyme furthermore comprises an amino acid substitution at a position corresponding to residue 434 in the polypeptide according to SEQ ID NO:1, particularly substitution of methionine at a position corresponding to M434 in SEQ ID NO:1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 35 in the polypeptide according to SEQ ID NO:1 leading to substitution of the original residue by any other amino acid except tyrosine, particularly through exchange of the original amino acid by phenylalanine, such as particularly via substitution of tyrosine by phenylalanine (e.g. Y35F in SEQ ID NO:1). Said enzyme to be modified can be derived from an ATF with at least about 20% identity to SbATF shown in SEQ ID NO:1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO:1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 346 in the polypeptide according to SEQ ID NO:1 leading to substitution of the original residue by any other amino acid except phenylalanine, particularly through exchange of the original amino acid by leucine, such as particularly via substitution of phenylalanine by leucine (e.g. F346L in SEQ ID NO:1). Said enzyme to be modified can be derived from an ATF with at least about 20% identity to SbATF shown in SEQ ID NO:1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO: 1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 371 in the polypeptide according to SEQ ID NO:1 leading to substitution of the original residue by any other amino acid except valine, particularly through exchange of the original amino acid by isoleucine, such as particularly via substitution of valine by isoleucine (e.g. V371I in SEQ ID NO:1). Said enzyme to be modified can be derived from an ATF with at least about 20% identity to SbATF shown in SEQ ID NO: 1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO:1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 373 in the polypeptide according to SEQ ID NO:1 leading to substitution of the original residue by any other amino acid except phenylalanine, particularly through exchange of the original amino acid by alanine, such as particularly via substitution of phenylalanine by alanine (e.g. F373A in SEQ ID NO:1). Said enzyme to be modified can be derived from an ATF with at least about 20% identity to SbATF shown in SEQ ID NO:1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO: 1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 419 in the polypeptide according to SEQ ID NO:1 leading to substitution of the original residue by any other amino acid except asparagine, particularly through exchange of the original amino acid by leucine, such as particularly via substitution of asparagine by leucine (e.g. N419L in SEQ ID NO:1). Said enzyme to be modified can be derived from an ATF with at least about 20% identity to SbATF shown in SEQ ID NO:1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO: 1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 434 in the polypeptide according to SEQ ID NO:1 leading to substitution of the original residue by any other amino acid except methionine, particularly through exchange of the original amino acid by valine, such as particularly via substitution of methionine by valine (e.g. M434V in SEQ ID NO:1). Said enzyme to be modified can be derived from an ATF with at least about 20% identity to SbATF shown in SEQ ID NO:1. Preferably, such modified enzyme furthermore comprises an amino acid substitution at a position corresponding to residue 34 in the polypeptide according to SEQ ID NO:1, particularly substitution of leucine at a position corresponding to L34 in SEQ ID NO:1 by phenylanaline. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 437 in the polypeptide according to SEQ ID NO:1 leading to substitution of the original residue by any other amino acid except arginine, particularly through exchange of the original amino acid by valine, such as particularly via substitution of arginine by valine (e.g. R437V in SEQ ID NO:1). Said enzyme to be modified can be derived from an ATF with at least about 20% identity to SbATF shown in SEQ ID NO: 1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO:1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

In one embodiment, the modified ATF enzyme as defined herein comprises an amino acid substitution at a position corresponding to residue 478 in the polypeptide according to SEQ ID NO:1 leading to substitution of the original residue by any other amino acid except tryptophane, particularly through exchange of the original amino acid by phenylalanine, such as particularly via substitution of tryptophane by phenylalanine (e.g. W478F in SEQ ID NO:1). Said enzyme to be modified can be derived from an ATF with at least about 20% identity to SbATF shown in SEQ ID NO:1. Preferably, such modified enzyme furthermore comprises two amino acid substitutions at positions corresponding to residues 34 and 434 in the polypeptide according to SEQ ID NO:1, particularly substitution of leucine at a position corresponding to L34 in SEQ ID NO:1 by phenylalanine and substitution of methionine at a position corresponding to M434 in SEQ ID NO:1 by valine. Using such modified enzyme comprising said mutation in a fermentation process using a suitable carbon source such as e.g. glucose results in an increase in retinyl acetate from acetylation of retinol of at least about 10 to more than 700% compared to the corresponding process using the respective wild-type enzyme, e.g. an enzyme according to SEQ ID NO:1.

As used herein, the term “original amino acid” refers to the amino acid residue present in the wild-type or non-modified ATF that is to be substituted by another amino acid residue in such a way that the activity of said modified enzyme towards acetylation of retinol is increased as defined herein.

The present invention is directed to a process for introduction of at least about 1 to 9, such as e.g. at least 2, amino acid substitution(s) into an ATF, particularly fungal ATF, e.g. an ATF according to SEQ ID NO:1 or with at least about 20% identity thereto, wherein the activity of said non-modified ATF as defined herein is increased by at least about 10%, i.e. acetylation of retinol into retinyl acetate that is increased by at least about 10% (to result in production of up to 86 wt % retinyl acetate based on total retinoids) as compared to percentage of retinyl acetate in the range of up to about 10 wt % obtained in a system/host cell using the respective non-modified ATF, such as e.g. ATF according to SEQ ID NO:1, wherein the single amino acid substitutions as described herein might be combined with one or more amino acid substitution(s) in order to increase the activity of the enzyme towards acetylation of retinol into retinyl acetate.

Thus, in some embodiments, the modified enzyme as described herein comprises 2 amino acid substitutions, particularly said amino acid substitutions being located on residues corresponding to position L34 and M434 in the polypeptide according to SEQ ID NO:1, more particularly comprising introduction of phenylalanine on a position corresponding to L34 and valine on a position corresponding to M434 in the polypeptide according to SEQ ID NO:1, such as amino acid substitutions corresponding to L34F and M434V in the polypeptide according to SEQ ID NO:1. The use of such modified enzyme can lead to an increase of retinyl acetate by at least about 150% as compared to percentage of retinyl acetate using the respective non-modified ATF, e.g. an enzyme according to SEQ ID NO:1.

In some embodiments, the modified enzyme as described herein comprises 3 amino acid substitutions, particularly said amino acid substitutions being located on residues corresponding to position L34 and M434 that are combined with an amino acid substitution on a position corresponding to F346, V371, N419, R437 or W478 in the polypeptide according to SEQ ID NO:1, more particularly comprising introduction of phenylalanine on a position corresponding to L34 and valine on a position corresponding to M434 in the polypeptide according to SEQ ID NO:1 in combination with introduction of leucine on a position corresponding to F346, isoleucine on a position corresponding to V371, leucine on a position corresponding to N419 or valine on a position corresponding to R437 in the polypeptide according to SEQ ID NO:1, such as amino acid substitutions corresponding to L34F and M434V combined with F346L, V371I, N419L or R437V in the polypeptide according to SEQ ID NO:1. The use of such modified enzymes can lead to an increase of retinyl acetate by at least about 60 to 550% as compared to percentage of retinyl acetate using the respective non-modified ATF, e.g. an enzyme according to SEQ ID NO:1.

In some embodiments, the modified enzyme as described herein comprises 4 amino acid substitutions, particularly said amino acid substitutions being located on residues corresponding to position L34 and M434 that are combined with an amino acid substitution on a position corresponding to Y35, F346, V371, N419, R437 or W478 in the polypeptide according to SEQ ID NO:1, more particularly comprising introduction of phenylalanine on a position corresponding to L34 and valine on a position corresponding to M434 in the polypeptide according to SEQ ID NO:1 in combination with introduction of phenylalanine on a position corresponding to Y35, leucine on a position corresponding to F346, isoleucine on a position corresponding to V371, leucine on a position corresponding to N419, valine on a position corresponding to R437 or phenylalanine on a position corresponding to W478 in the polypeptide according to SEQ ID NO:1, such as amino acid substitutions corresponding to L34F and M434V combined with Y35F, F346L, V371I, N419L R437V or W478F in the polypeptide according to SEQ ID NO:1, including but not limited to amino acid substitutions L34F_Y35F_V371I_M434V or L34F_V371I_M434V_N419L or L34F_V371I_M434V_F346L or L34F_M434V_N419L_F346L or L34F_V371I_M434V_F437V or L34F_M434V_N419L_R437V or L34F_M434V_N419L_W478F. The use of such modified enzymes can lead to an increase of retinyl acetate by at least about 230 to 670% % as compared to percentage of retinyl acetate using the respective non-modified ATF, e.g. an enzyme according to SEQ ID NO:1.

In some embodiments, the modified enzyme as described herein comprises 5 amino acid substitutions, particularly said amino acid substitutions being located on residues corresponding to position L34, M434 and V371 that are combined with an amino acid substitution on a position corresponding to Y35, F346, N419, R437 or W478 in the polypeptide according to SEQ ID NO:1, more particularly comprising introduction of phenylalanine on a position corresponding to L34 and valine on a position corresponding to M434 in the polypeptide according to SEQ ID NO:1 in combination with introduction of phenylalanine on a position corresponding to Y35, leucine on a position corresponding to F346, leucine on a position corresponding to N419, valine on a position corresponding to R437 or phenylalanine on a position corresponding to W478 in the polypeptide according to SEQ ID NO:1, such as amino acid substitutions corresponding to L34F and M434V combined with Y35F, F346L, N419L, R437V or W478F in the polypeptide according to SEQ ID NO:1, including but not limited to amino acid substitutions L34F_Y35F_V371I_M434V_N419L or L34F_V371I_M434V_N419L_F436L or L34F_Y35F_V371I_M434V_R437V or L34F_V371I_M434V_N419L_R437V or L34F_Y35F_V371I_M434V_W478F or L34F_V371I_M434V_N419L_W478F. The use of such modified enzymes can lead to an increase of retinyl acetate by at least about 300 to 760% as compared to percentage of retinyl acetate using the respective non-modified ATF, e.g. an enzyme according to SEQ ID NO:1.

In some embodiments, the modified enzyme as described herein comprises 6 amino acid substitutions, particularly said amino acid substitutions being located on residues corresponding to position L34, Y35, V371, M434 and N419 that are combined with an amino acid substitution on a position corresponding to F346, R437 or W478 in the polypeptide according to SEQ ID NO:1, more particularly comprising introduction of phenylalanine on a position corresponding to L34, phenylalanine on a position corresponding to Y35, isoleucine on a position corresponding to V371, valine on a position corresponding to M434 and leucine on a position corresponding to N419 in the polypeptide according to SEQ ID NO:1 in combination with introduction of leucine on a position corresponding to F346, valine on a position corresponding to R437 or phenylalanine on a position corresponding to W478 in the polypeptide according to SEQ ID NO:1, such as amino acid substitutions corresponding to L34F, Y35F, V371I, M434V and N419L combined with F346L, R437V or W478F in the polypeptide according to SEQ ID NO:1, including but not limited to amino acid substitutions L34F_Y35F_V371I_M434V_N419L_F346L or L34F_Y35F_V371I_M434V_N419L_R437V or L34F_Y35F_V371I_M434V_N419L_W478F. The use of such modified enzymes can lead to an increase of retinyl acetate by at least about 130 to 600% as compared to percentage of retinyl acetate using the respective non-modified ATF, e.g. an enzyme according to SEQ ID NO:1.

In some embodiments, the modified enzyme as described herein comprises 8 amino acid substitutions, particularly said amino acid substitutions being located on residues corresponding to position L34, Y35, V371, M434, N419, F346, R437 and W478 in the polypeptide according to SEQ ID NO:1, more particularly comprising introduction of phenylalanine on a position corresponding to L34, phenylalanine on a position corresponding to Y35, isoleucine on a position corresponding to V371, valine on a position corresponding to M434, leucine on a position corresponding to N419, leucine on a position corresponding to F346, valine on a position corresponding to R437 and phenylalanine on a position corresponding to W478 in the polypeptide according to SEQ ID NO:1, such as amino acid substitutions corresponding to L34F_Y35F_V371I_M434V_N419L_F346L_R437V_W478F. The use of such modified enzymes can lead to an increase of retinyl acetate by at least about 700% as compared to percentage of retinyl acetate using the respective non-modified ATF, e.g. an enzyme according to SEQ ID NO:1.

In some embodiments, the modified enzyme as described herein comprises 9 amino acid substitutions, particularly said amino acid substitutions being located on residues corresponding to position L34, Y35, V371, M434, N419, F346, R437, W478 and F373 in the polypeptide according to SEQ ID NO:1, more particularly comprising introduction of phenylalanine on a position corresponding to L34, phenylalanine on a position corresponding to Y35, isoleucine on a position corresponding to V371, valine on a position corresponding to M434, leucine on a position corresponding to N419, leucine on a position corresponding to F346, valine on a position corresponding to R437, phenylalanine on a position corresponding to W478 and alanine on a position corresponding to F373 in the polypeptide according to SEQ ID NO:1, such as amino acid substitutions corresponding to L34F_Y35F_V371I_M434V_N419L_F346L_R437V_W478F_F373A. The use of such modified enzymes can lead to an increase of retinyl acetate by at least about 10% as compared to percentage of retinyl acetate using the respective non-modified ATF, e.g. an enzyme according to SEQ ID NO:1.

The modified host cell as described herein is capable of conversion of retinol into retinyl acetate with conversion increased by at least about 10% or more compared to conversion via the respective non-modified enzyme, e.g. polypeptide according to SEQ ID NO:1, particularly with an increase in retinyl acetate production by 10 to 760% and more with a percentage of retinyl acetate based on total retinoids in the range of up to 86 wt %, such as e.g. obtainable via expression of a modified ATF under suitable culture conditions including but not limited to cultivation on glucose, galactose, xylose with or without the presence of ethanol, and a suitable host cell, such as e.g. selected from a fungal host cell including Yarrowia or Saccharomyces or other microbial host cell such as e.g. Escherichia coli. Suitable conditions might be cultivation in a fed-batch fermentation of e.g. 80, 90, 100, 110, 120, 130 h.

A modified host cell as defined herein comprises one or more copies of modified ATFs as defined herein, preferably wherein the ATFs are heterologous expressed in said modified host cell. Modifications in order to have the host cell as defined herein produce more copies of genes and/or proteins, such as e.g. more copies of modified ATFs with selectivity towards formation of retinyl acetate as defined herein, including increased conversion of retinol into retinyl acetate by at least about 10% as compared to a process using the respective non-modified ATF, such as e.g. SbATF1 according to SEQ ID NO:1 (FIG. 1), such as e.g. obtainable via expression of a modified ATF under suitable culture conditions including but not limited to cultivation on glucose, galactose, xylose with or without the presence of ethanol, may include the use of strong promoters, suitable transcriptional—and/or translational enhancers, or the introduction of one or more gene copies into the retinol-producing host cell, particularly fungal host cell, leading to increased accumulation of the respective enzymes in a given time. The skilled person knows which techniques to use depending on the host cell. The increase or reduction of gene expression can be measured by various methods, such as e.g. Northern, Southern or Western blot technology as known in the art.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention is directed to a retinol-producing host cell, particularly fungal host cell, as described herein comprising an expression vector or a polynucleotide encoding modified ATFs, as described herein which has been integrated in the chromosomal DNA of the host cell. Such retinol-producing host cell, particularly fungal host cell, comprising a heterologous polynucleotide either on an expression vector or integrated into the chromosomal DNA encoding modified ATFs as described herein is called a recombinant or modified host cell. The retinol-producing host cell, particularly fungal host cell, might contain one or more copies of a gene encoding the modified ATFs as defined herein, comprising the mutations as defined herein, leading to overexpression of such genes encoding said modified ATFs, as defined herein. The increase of gene expression can be measured by various methods, such as e.g. Northern, Southern or Western blot technology as known in the art. The present invention is particularly directed to the use of such novel modified ATF enzymes, in a process for production of retinyl acetate, particularly under conditions wherein the amount of other retinyl esters, particularly long-chain retinyl esters is reduced. The skilled person knows how to generate such conditions (see, e.g. WO2021136689 or WO2022090548). Retinyl acetate can be further converted into vitamin A by the action of (known) suitable chemical or biotechnological mechanisms.

The terms “sequence identity”, “% identity” are used interchangeable herein. For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The modified ATF enzymes as defined herein also encompass enzymes carrying further amino acid substitution(s) which do not alter enzyme activity, i.e. which show the same properties with respect to the enzymes defined herein and catalyze the conversion of retinol to retinyl acetate with formation of about 86 wt % or more (e.g. 11 to 86 wt %) retinyl acetate based on the total amount of retinoids. Such mutations are also called “silent mutations”, which do not alter the (enzymatic) activity of the enzymes according to the present invention.

Expression of the enzymes/polynucleotides encoding one of the modified enzymes, as defined herein can be achieved in any host system, including (micro) organisms, which is suitable for retinoid (including retinol) production and which allows expression of the nucleic acids encoding one of the enzymes as disclosed herein, including functional equivalents or derivatives as described herein. Examples of suitable retinol-producing host (micro) organisms are bacteria, algae, fungi, including yeasts, plant or animal cells. Preferred bacteria are those of the genera Escherichia, such as, for example, Escherichia coli, Streptomyces, Pantoea (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Kluyveromyces, such as Kluyveromyces lactis, Phycomyces, such as Phycomyces blakesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

Depending on the host cell the polynucleotides as defined herein for acetylation of retinol might be optimized for expression in the respective host cell. The skilled person knows how to generate such further modified polynucleotides. It is understood that the polynucleotides as defined herein also encompass such host-optimized nucleic acid molecules as long as they still express the polypeptide with the respective activities as defined herein.

Thus, in one embodiment, the present invention is directed to a retinol-producing host cell, particularly fungal host cell, comprising polynucleotides encoding modified ATF enzymes as defined herein which are optimized for expression in said host cell. Particularly, a retinol-producing host cell, particularly fungal host cell, is selected from yeast, e.g. Yarrowia or Saccharomyces, such as e.g. Saccharomyces cerevisiae or Yarrowia lipolytica, wherein the polynucleotides encoding the modified ATF enzymes as defined herein are selected from polynucleotides expressing modified polypeptides comprising one or more amino acid substitution(s) in a sequence with at least 20%, such as e.g. 25, 30, 35, 40, 45, 50, 55, 60, 65, 70, 75, 80, 85, 90, 92, 95, 97, 98, 99% or up to 100% identity to SEQ ID NO:1, such as e.g. introduction of one or more amino acid substitution(s) at position(s) corresponding to residue(s) selected from the group consisting of position L34, Y35, V371, M434, N419, F346, R437, W478, F373, and combinations thereof and as defined herein and preferably comprising a highly conserved partial amino acid sequence, i.e. common active site or “Prosite-motif”, of at least 7 amino acid residues selected from [NSDEHC]-H-x (3)-D-[GA] corresponding to position N190 to G196 in the polypeptide according to SEQ ID NO:1 (motifs are in Prosite syntax, as defined in https://prosite.expasy.org/scanprosite/scanprosite_doc.html) and wherein “x” denotes an arbitrary amino acid, said host cell producing retinyl acetate with an increase of at least about 10% compared to a host cell expressing the respective non-modified enzyme, e.g. an enzyme according to SEQ ID NO:1, such as e.g. obtainable via expression of a modified ATF under suitable culture conditions including but not limited to cultivation on glucose, galactose or xylose.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The present invention is directed to a process for production of retinyl acetate, wherein the retinyl acetate is generated via acetylation of retinol (particularly at least 65% as trans-retinol) as disclosed herein by the action of modified ATF enzymes as described herein, wherein the acetylating enzymes are preferably heterologous expressed in a suitable host cell under suitable conditions as described herein. The produced retinyl acetate might be isolated and optionally further purified from the medium and/or host cell. Said acetylated retinoids defined herein can be used as building blocks in a multi-step process leading to vitamin A. Vitamin A might be isolated and optionally further purified from the medium and/or host cell as known in the art.

Preferably, acetylation of retinol by the use of modified ATFs as described herein, can lead to retinyl acetate percentage in the range of at least about 11 to 86 wt % based on total retinoids, i.e. retinyl acetate present in the retinoid mix produced by the host cell, such as e.g. obtainable via expression of a modified enzymes under suitable culture conditions including but not limited to cultivation on glucose, galactose or xylose. In a more preferred embodiment, a retinol mix with a percentage of at least about 65% trans-retinol is used as substrate for acetylation via the modified enzymes as defined herein.

The host cell, i.e. microorganism, algae, fungal, animal or plant cell, capable of producing retinol, might furthermore be capable of production of beta-carotene, which might be furthermore enzymatically converted into retinal which might be furthermore converted into retinol. The skilled person knows which genes to be used/expressed for either biosynthesis of beta-carotene and/or bio-conversion of beta-carotene into retinol. Such host cell further being capable of expressing the modified ATFs as defined herein, and/or further genes required for biosynthesis of vitamin A, may be cultured in an aqueous medium supplemented with appropriate nutrients under aerobic or anaerobic conditions and as known by the skilled person for the respective retinol-producing host cells. Optionally, such cultivation is in the presence of proteins and/or co-factors involved in transfer of electrons, as known in the art. Suitable carbon sources for the purpose of the present invention might be selected from glucose, fructose, raffinose, lactose, galactose, glycerol, xylose, arabinose, sucrose or maltose with or without the presence of ethanol, particularly selected from glucose, galactose or xylose. Particular culture conditions might contain a batch and feed run, with a concentration of 5% (w/v) glucose and 1% ethanol (w/v) in the batch phase and a concentration of 100% (w/v) in the feeding phase. The cultivation/growth of the host cell may be conducted in batch, fed-batch, semi-continuous or continuous mode, particularly in fed-batch mode for 80, 90, 100, 110, 120, 130 h under suitable culture conditions. Depending on the host cell, preferably, production of retinoids such as e.g. vitamin A, precursors and/or derivatives thereof such as retinal, retinol, retinyl esters, particularly retinyl acetate, can vary, as it is known to the skilled person. Cultivation and isolation of beta-carotene and retinoid-producing host cells selected from Yarrowia and Saccharomyces is described in e.g. WO2008042338. With regards to production of beta-carotene and retinoids in host cells selected from E. coli, methods are described in e.g. US20070166782.

Particularly, the fermentation using suitable retinoid-producing host strains as defined herein expressing the modified ATFs as described herein are cultivated in a two-phase system, wherein the retinoids, including but not limited to retinyl acetate, are collected in and afterwards isolated from a suitable lipophilic phase. Particular conditions and lipophilic solvents are disclosed in WO2022090548 or WO2022090549.

In some embodiments, the present invention is directed to a two-phase fermentation using a lipophilic solvent as second phase, such as e.g. isopars or corn oil, besides the known solvents comprising Drakeol®, silicone or n-dodecane (see Jang et al., Microbial Cell Factories 10:59, 2011).

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity, Analytical methods to evaluate the capability of a suitable ATF, particularly Atf1, as defined herein for retinyl acetate production, from conversion of retinol are known in the art, such as e.g. described in Example 4 of WO2014096992. In brief, titers of products such as retinyl acetate, retinol, trans-retinal, cis-retinal, beta-carotene and the like can be measured by HPLC.

With regards to suitable host cells comprising specific enzymes involved in biosynthesis of beta-carotene and that are expressed and active in vivo leading to production of carotenoids, e.g. beta-carotene, both genes and methods to generate carotenoid-producing host cells are known in the art, see e.g. WO2006102342. Depending on the carotenoid to be produced, different genes might be involved.

As used herein, a “retinol-producing host cell” is a host cell, wherein the respective polypeptides are expressed and active in vivo, leading to production of retinoids, e.g. vitamin A and its precursors including retinol, via enzymatic conversion of beta-carotene via retinal into retinol. These polypeptides include the modified ATFs as defined herein. The genes of the vitamin A pathway and methods to generate retinoid-producing host cells are known in the art. The term retinoid includes retinol, which is used as a substrate for the modified acetylating enzymes as defined herein.

Retinoids as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. Long chain retinyl esters as used herein are defined as hydrocarbon esters of retinol with fatty acids, where the fatty acids consist of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. The fatty acids in the long chain retinyl esters include but are not limited to linoleic acid, oleic acid or palmitic acid. Biosynthesis of retinoids is described in e.g. WO2008042338.

“Retinal” as used herein is known under IUPAC name (2E,4E,6E,8E)-3,7-Dimethyl-9-(2,6,6-trimethylcyclohexen-1-yl) nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

Particularly, the present invention is directed to the following embodiments (1) to (14):

(1) Method for modification of an enzyme catalyzing the acetylation of retinoids, particularly retinol into retinyl acetate, preferably a fungal enzyme, comprising introduction of at least 1 amino acid substitution(s) at one or more position(s) selected from the group consisting of amino acid residue 34, 35, 346, 371, 373, 419, 434, 437, 478, and combinations thereof in the polypeptide according to SEQ ID NO: 1, wherein upon introduction of the one or more amino acid substitution(s) the activity towards formation of retinyl acetate is increased by at least about 10%, such as in the range of 10 to 760% compared to the activity using the respective non-modified enzyme including an enzyme according to SEQ ID NO:1.

(2) Method according to embodiment (1), wherein the amino acid residue to be substituted on position corresponding to position 34 in SEQ ID NO:1 is not leucine, and/or wherein the amino acid residue to be substituted on position corresponding to position 35 in SEQ ID NO:1 is not tyrosine, and/or wherein the amino acid residue to be substituted on position corresponding to position 346 or 373 in SEQ ID NO:1 is not phenylalanine, and/or wherein the amino acid residue(s) to be substituted on position corresponding to position 371 in SEQ ID NO: 1 is not valine, and/or wherein the amino acid residue(s) to be substituted on position corresponding to position 419 in SEQ ID NO:1 is not asparagine, and/or wherein the amino acid residue(s) to be substituted on position corresponding to position 434 in SEQ ID NO:1 is not methionine, and/or wherein the amino acid residue(s) to be substituted on position corresponding to position 437 in SEQ ID NO:1 is not arginine, and/or wherein the amino acid residue(s) to be substituted on position corresponding to position 478 in SEQ ID NO: 1 is not tryptophane.

(3) Method according to embodiment (1) or (2), wherein the amino acid residue to be introduced on position corresponding to position 34, 35 or 478 in SEQ ID NO: 1 is phenylalanine, and/or wherein the amino acid residue to be introduced on position corresponding to position 346 or 419 in SEQ ID NO:1 is leucine, and/or wherein the amino acid residue(s) to be introduced on position corresponding to position 371 in SEQ ID NO:1 is isoleucine, and/or wherein the amino acid residue(s) to be introduced on position corresponding to position 373 in SEQ ID NO:1 is alanine, and/or wherein the amino acid residue(s) to be introduced on position corresponding to position 434 or 437 in SEQ ID NO:1 is valine.

(4) Method according to embodiment (1), (2) or (3), wherein an enzyme with at least 20% identity to SEQ ID NO:1 is modified via introduction of one or more amino acid substitution(s) on position corresponding to amino acid residue(s) L34, Y35, F346, V371, F373, N419, M434, R437, W478, and/or combinations thereof, preferably wherein the modified enzyme comprises one or more amino acid substitution(s) selected from the group consisting of L34F, Y35F, V371I, M434V, N419L, F346L, R437V, W478F, F373A, and combinations thereof.

(5) Method according to embodiment (1), (2), (3) or (4), wherein upon introduction of the one or more amino acid substitution(s) the percentage of retinyl acetate based on total retinoids in a process comprising expression of said modified enzyme in a suitable host cell is increased to about 86 wt % compared to the percentage using the respective non-modified enzyme including an enzyme according to SEQ ID NO:1.

(6) Process for production of retinol acetylating enzymes, preferably acetyl transferase of class [EC 2.3.1.84], having increased activity towards acetylation of retinol into retinyl acetate, said process comprising:
(a) providing a fungal enzyme involved in acetylation of retinol, particularly ATF originated from Saccharomyces, wherein said non-modified enzyme expressed in a suitable host cell and under suitable culture conditions is capable of producing up to 10 wt % retinyl acetate from retinol based on total retinoids produced by said host cell;
(b) introduction of 1 or more amino acid substitution(s), such as at least 1 to 9 amino acid substitution(s) into the enzyme of (a) to generate a modified enzyme involved in acetylation of retinol, wherein the ability to convert retinol into retinyl acetate is increased by at least about 10%, such as e.g. by at least 10 to 760% when compared to the acetylation of the respective non-modified enzyme of step (a);
wherein the 1 or more amino acid substitution(s) are being located at position(s) corresponding to amino acid residue(s) being selected from the group consisting of position 34, 35, 346, 371, 373, 419, 434, 437, 478 in SEQ ID NO:1 and wherein the substitute amino acid(s) is/are different from L34, Y35, F346, V371, F373, N419, M434, R437, W478 and/or combinations thereof, wherein the amino acid residue to be introduced at position corresponding to position 34, 35, or 478 in SEQ ID NO: 1 is preferably phenylalanine, and/or wherein the amino acid residue to be introduced at position corresponding to position 346 or419 in SEQ ID NO:1 is preferably leucine, and/or wherein the amino acid residue to be introduced at position corresponding to position 371 in SEQ ID NO:1 is preferably isoleucine, and/or wherein the amino acid residue to be introduced at position corresponding to position 373 in SEQ ID NO:1 is preferably alanine, and/or
wherein the amino acid residue to be introduced at position corresponding to position 434 or 437 in SEQ ID NO:1 is preferably valine.

(7) Process according to embodiment (6), wherein the acetyl transferase of step (a) is selected from yeast ATF.

(8) Modified enzyme involved in acetylation of retinol into retinyl acetate, particularly fungal enzyme comprising one or more modification(s), such as at least 1, 2, 3, 4, 5, 6, 7, 8, 9 amino acid substitution(s), said amino acid substitution(s) being located at position(s) corresponding to amino acid residue(s) being selected from the group consisting of position 34, 35, 346, 371, 373, 419, 434, 437, 478 and combinations thereof, in the polypeptide according to SEQ ID NO:1 or a polypeptide with a sequence of at least about 20%, such as 25, 30, 35, 40, 45, 50, 55, 60, 65, 70, 75, 80, 85, 90, 92, 95, 97, 98, 99% or up to 100% identity to SEQ ID NO:1, and wherein the substitute amino acid residue(s) are different from amino acids corresponding to L34, Y35, F346, V371, F373, N419, M434, R437, W478 and combinations thereof in the enzyme according to SEQ ID NO: 1, preferably wherein the amino acid residue(s) to be substituted are corresponding to residues selected from the group consisting of L34, Y35, F346, V371, F373, N419, M434, R437, W478 and combinations thereof in a polypeptide according to SEQ ID NO:1, particularly wherein the substitute amino acid residue to be introduced at position corresponding to position 34, 35, or 478 in SEQ ID NO: 1 is preferably phenylalanine, wherein the substitute amino acid residue to be introduced at position corresponding to position 346 or419 in SEQ ID NO:1 is preferably leucine, wherein the substitute amino acid residue to be introduced at position corresponding to position 371 in SEQ ID NO:1 is preferably isoleucine, wherein the substitute amino acid residue to be introduced at position corresponding to position 373 in SEQ ID NO:1 is preferably alanine, and/or wherein the substitute amino acid residue to be introduced at position corresponding to position 434 or 437 in SEQ ID NO:1 is preferably valine.

(9) Modified enzyme according to embodiment (8) selected from a polypeptide with at least about 20%, such as 25, 30, 35, 40, 45, 50, 55, 60, 65, 70, 75, 80, 85, 90, 92, 95, 97, 98, 99% or up to 100% identity to SEQ ID NO:1, comprising one or more amino acid substitution(s) on position(s) selected from 34, 35, 346, 371, 373, 419, 434, 437 and/or 478 in SEQ ID NO:1 being different from L34, Y35, F346, V371, F373, N419, M434, R437 or W478, preferably wherein the one or more amino acid substitution(s) are selected from the group consisting of L34F, Y35F, V371I, M434V, N419L, F346L, R437V, W478F, F373A, and combinations thereof.

(10) Modified enzyme according to embodiment (8) or (9), wherein the enzymatic activity towards formation of retinyl acetate is increased by at least about 10% compared to the respective non-modified ATF, e.g. ATF according to SEQ ID NO:1, particularly with an increase in the percentage of retinyl acetate based on total retinoids to about 86 wt %.

(11) Modified enzyme according to embodiment (8), (9) or (10) comprising amino acid substitution corresponding to position 34 and 434 in SEQ ID NO:1, preferably wherein the amino acid substitution corresponds to L34F_M434V in the polypeptide according to SEQ ID NO:1.

(12) Process for production of retinyl acetate, comprising the step of acetylation of retinol via action of an enyzme according to embodiment (8), (9), (10) or (11).

(13) Process according to embodiment (12), wherein a suitable retinol producing host cell, preferably selected from Yarrowia, Saccharomyces or Escherichia coli, is transformed with and expressing an enzyme according to embodiment (8), (9), (10) or (11).

(14) Process according to embodiment (12) or (13), wherein the percentage of retinyl acetate produced under suitable culture conditions is about 86 wt % based on total retinoids produced by said host cell.

### FIGURES

FIG. 1: Amino acid sequence of Saccharomyces bayanus ATF1 (SbATF; SEQ ID NO: 1) wherein the residues selected for the amino acid substitutions as described in the present application are marked in bold/underlined.

The following examples are illustrative only and are not intended to limit the scope of the invention in any way. The contents of all references, patent applications, patents, and published patent applications, cited throughout this application are hereby incorporated by reference, in particular WO2014096992, WO2019058001, WO2021136689, WO2022090548, WO2008042338, US20070166782, WO2022090549, WO2006102342, WO2020141168, and WO2016172282.

### EXAMPLES

### Example 1: General Methods, Strains, and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay (Yarrowia). For testing the conversion activity of the mutants, typically, 200 μl of 0.25% Yeast extract, 0.5% peptone (0.25×YP) was inoculated with 10 μl of freshly grown Yarrowia and overlaid with 200 μl of mineral oil (Isopar M, Exxon Mobile) with 2% oleic acid as carbon source in the mineral oil phase. Transformants were grown in 24 well plates (Microplate Devices 24 Deep Well Plates Whatman 7701-5102), covered with mat seal (Analytical Sales and Services Inc. Plate Mats 24010 CM), sterile sealed with Qiagen Airpore Tape Sheets (19571) and shaken in Infors multi plate shaker (Multitron), 30° C., 800 RPM for 4 days. The mineral oil fraction was removed from the shake plate wells and analyzed by UPLC reverse phase column, with a photo-diode array detector. This method is also used in Example 2.

DNA transformation. Yarrowia lipolytica strains were transformed from overnight growth on YPD plate media. 50 μl of cells was scraped from a plate and transformed by incubation in 500 μl with 1 μg transforming DNA, typically linear DNA for integrative transformation, 40% PEG 3550 MW, 100 mM lithium acetate, 50 mM Dithiothreitol, 5 mM Tris-Cl pH 8.0, 0.5 mM EDTA for 30 minutes at 40° C. and plated directly to selective media or, in the case of dominant antibiotic marker selection, the cells were out grown on YPD liquid media for 4 hours at 30° C. before plating on the selective media. Saccharomyces strains were transformed using the lithium-acetate method from exponential phase YPD-grown cells, which were grown by subculture of an overnight YPD culture. 10⁸ cells/transformation were harvested and resuspended in a mixture containing 40% PEG 3350 (MW), 100 mM lithium acetate, 10 mM Tris-Cl pH 8.0, 1 mM EDTA, 5 μg sheared salmon sperm DNA, and 2 μg of linearized transforming DNA, in a final volume of 500 μL. This mixture was incubated at 30° C. for 1 hour, followed by 42° C. for 30 minutes. The cells were then pelleted and resuspended in liquid YPD media and permitted to grow for 3 hours at 30° C. or overnight at 22° C. to allow for expression of the HygR antibiotic resistance gene before plating on the selective media, containing 100 μg/ml hygromycin. Most of the DNA sequences used herein are codon-optimized for expression in the respective host system and as indicated in the sequence listing.

DNA molecular biology. Plasmid MB10362 (SEQ ID NO:3) containing expression systems for DrBCO, LmATF, and FfRDH was synthesized at Genscript (Piscataway, NJ, USA). Plasmid MB10362 contains both the ‘URA3’ and ‘HOM3” markers for selection in Yarrowia lipolytica transformations. For clean gene insertion by random nonhomologous end joining of the gene and marker Sfil plasmid fragment of MB10362, or other plasmids in Table 1 of interest was purified by gel electrophoresis and Qiagen gel purification column. Clones were verified by sequencing. Typically, genes are synthesized at GenScript (Piscataway, NJ), with introduction of amino acid substitutions according to Table 1. Transformants were screened for homoserine auxotrophy and subsequently sequenced using primers flanking the HOM3 sequence and clean frameshifts were selected to move forward. Expression of mutant ATFs in Saccharomyces cerevisiae is described in Example 1 of WO2020141168.

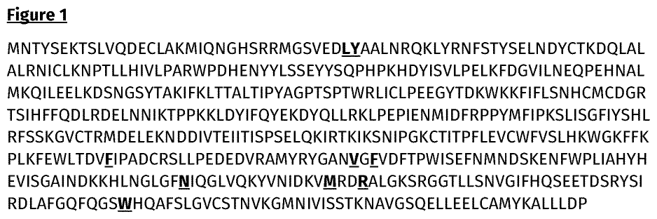

Sequences. Plasmids used for expression of the wild-type SbATF (polynucleotide according to SEQ ID NO:2) as well as the modified enzymes including specific amino acid substitutions are listed in Table 1 and/or the sequence listing, with codon-optimized sequences for expression in Yarrowia lipolytica or Saccharomyces cerevisiae specifically indicated. FIG. 1 shows the wild-type SbATF amino acid sequence with specific indication of the residues selected for modification according to Table 1.

TABLE 1
list of plasmids used for construction of the strains carrying the non-
modified or modified heterologous Yarrowia lipolytica codon-optimized
ATF-genes from Saccharomyces bayanus as insert. All inserts are based
on SbATF according to SEQ ID NO: 1. For more details, see text.

| Plasmid | Insert |
|---|---|
| MB10362 | SbATF |
| MB10363 | SbATF_L34F_M434V |
| MB10364 | SbATF_L34F_V371I_M434V |
| MB10365 | SbATF_L34F_Y35F_V371I_M434V |
| MB10366 | SbATF_L34F_M434V_N419L |
| MB10367 | SbATF_L34F_V371I_M434V_N419L |
| MB10368 | SbATF_L34F_Y35F_V371I_M434V_N419L |
| MB10369 | SbATF_L34F_M434V_F346L |
| MB10370 | SbATF_L34F_V371I_M434V_F346L |
| MB10372 | SbATF_L34F_M434V_N419L_F346L |
| MB10373 | SbATF_L34F_V371I_M434V_N419L_F346L |
| MB10374 | SbATF_L34F_Y35F_V371I_M434V_N419L_F346L |
| MB10381 | SbATF_L34F_M434V_R437V |
| MB10382 | SbATF_L34F_V371I_M434V_R437V |
| MB10383 | SbATF_L34F_Y35F_V371I_M434V_R437V |
| MB10384 | SbATF_L34F_M434V_N419L_R437V |
| MB10385 | SbATF_L34F_V371I_M434V_N419L_R437V |
| MB10386 | SbATF_L34F_Y35F_V371I_M434V_N419L_R437V |
| MB10387 | SbATF_L34F_M434V_W478F |
| MB10388 | SbATF_L34F_V371I_M434V_W478F |
| MB10389 | SbATF_L34F_Y35F_V371I_M434V_W478F |
| MB10390 | SbATF_L34F_M434V_N419L_W478F |
| MB10391 | SbATF_L34F_V371I_M434V_N419L_W478F |
| MB10392 | SbATF_L34F_Y35F_V371I_M434V_N419L_W478F |
| MB10393 | SbATF_L34F_Y35F_V371I_M434V_N419L_F346L_R437V_W478F |
| MB10394 | SbATF_L34F_Y35F_V371I_M434V_N419L_F346L_R437V_W478F_F373A |

UPLC reverse phase retinol method. For rapid screening this method does not separate cis-isomers, only major functional groups. A Waters Acquity UPLC with PDA detection (or similar) with auto sampler was used to inject samples. An Acquity UPLC HSS T3 1.8 μm P/N 186003539 was used to resolve retinoids. The mobile phase consisted of either, 1000 mL hexane, 30 mL isopropanol, and 0.1 mL acetic acid for retinoid related compounds. The flow rate for each was 0.6 mL per minute. Column temperature was 20° C. The injection volume was 5 μL. The detector was a photodiode array detector collecting from 210 to 600 nm. Analytes were detected according to Table 2.

TABLE 2A
list of analytes using reverse phase retinol method. The addition
of all added intermediates gives the total amount retinoids. Beta-
carotene* can be detected in 325 nm and will interfere with retinyl
ester quantitation, therefore care must be taken to observe the
carotene peak and not include them in the retinoid quantification.
“N/A” means “not available”. For more details, see text.

| | Retention | Lambda max | Response |
|---|---|---|---|
| Intermediates | time [min] | [nm] | factor |
| retinyl-acetate | 2.93 | 325 | 1.00 |
| retinyl-esters | 3.2-3.8 | 325 | 1.68 |
| retinal | 2.77 | 325 | 0.87 |
| retinol | 2.73 | 325 | 0.87 |
| Beta-carotene* | 3.56 | 450 | N/A |

TABLE 2B
UPLC Method Gradient with solvent A: water; solvent B: acetonitrile;
solvent C: methanol; solvent D: tert-butyl methyl ether.

| Time | | | | | Flow | Pressure |
|---|---|---|---|---|---|---|
| [min] | % A | % B | % C | % D | [ml/min] | [psi/bar] |
| 0 | 50 | 50 | 0 | 0 | 0.5 | 9500-14000max |
| 0.5 | 50 | 50 | 0 | 0 | 0.5 | |
| 1.0 | 0 | 50 | 50 | 0 | 0.5 | |
| 1.25 | 0 | 0 | 100 | 0 | 0.5 | |
| 3.25 | 0 | 0 | 5 | 95 | 0.5 | |
| 3.5 | 0 | 0 | 5 | 95 | 0.5 | |
| 4.0 | 0 | 0 | 100 | 0 | 0.5 | |
| 4.25 | 0 | 50 | 50 | 0 | 0.5 | |
| 4.5 | 50 | 50 | 0 | 0 | 0.5 | |

Method Calibration. Method is calibrated on retinyl acetate, retinols and retinals are quantitated against retinyl-acetate using the indicated response factor. Retinyl Acetate is dissolved in THF at ~200 μg/ml for stock solution using a volumetric flask. Using volumetric flasks, ×20, ×50 and ×100 dilutions of stock solution in 50/50 methanol/MTBE were made. UV absorbance of retinyl acetate becomes nonlinear fairly quickly, so care must be taken to stay within the linear range. Consequently, lower concentrations might be better. Retinyl palmitate can also be used as retinyl ester calibration. Peaks for retinyl acetate at about 3 minutes and peaks for retinyl esters (long-chain retinyl esters) at around 3.5 minutes.

Sample preparation. Samples were prepared by various methods depending on the conditions. For whole broth or washed broth samples the broth was placed in a Precellys® tube, weighed, and mobile phase was added. Briefly in a 2 ml Precellys® tube, add 25 μl of well mixed broth and 975 μl of THF. The samples were then processed in a Precellys® homogenizer (Bertin Corp, Rockville, MD, USA) on the highest setting 3× according to the manufacturer's directions, typically 3×15×7500 tpms. For the washed pellet the samples were spun in a 1.7 ml tube in a microfuge at 10000 rpm for 1 minute, the broth decanted, 1 ml water added, mixed, pelleted and decanted, and brought up to the original volume. The mixture was pelleted again and brought up in appropriate amount of mobile phase and processed by Precellys® bead beating. For analysis of silicone oil fraction, the sample was spun at 4000 RPM for 10 minutes and the oil was decanted off the top by positive displacement pipet (Eppendorf, Hauppauge, NY, USA) and diluted into mobile phase mixed by vortexing and measured for retinoid concentration by UPLC analysis.

Fermentation conditions in Yarrowia. Fermentations were identical to the previously described conditions using preferably a silicone oil overlay and stirred tank that was preferably glucose in a bench top reactor with 0.5 L to 5 L total volume (see WO2016172282). Generally, the same results were observed with a fed batch stirred tank reactor with an increased productivity demonstrating the utility of the system for the production of retinoids. Preferably, fermentations were batched with 5% glucose and 20% silicone oil was added after dissolved oxygen dropped below about 20% and feed was resumed to achieve 20% dissolved oxygen throughout the feeding program.

### Example 2: Production of Retinyl Acetate in Yarrowia lipolytica Expressing Mutant SbATF

For expression of heterologous ATF in Yarrowia lipolytica as a host, the strain ML15710 (see Ex. 5 in WO2016172282) was transformed with plasmid MB9287 (see Ex. 1 in WO2022090548) to isolate a lip2 lip3 lip8 mutant derivative. This derivative was selected on 5-fluoorotic acid to isolate an uracil auxotroph, designated as strain ML18667-new. This strain was transformed with a plasmid listed in Table 1 above, each of which consists of the indicated ATF allele, DrBCO, and FfRDH12. Transformants of ML18667-new with Sfil-linearized plasmids from Table 1 were selected for uracil prototrophy. Transformants were grown in shake plates as described in Example 1, and the percentage of retinyl acetate (retinyl acetate/total retinoids) using the mutant ATFs in relation to the percentage of retinyl acetate using the reference SbATF expressed on plasmid MB10362 (set at 100%) is shown in Table 3.

TABLE 3A
acetylation of retinol into retinyl acetate (“retAc”)
as enhanced by action of modified ATFs comprising 2 amino
acid substitutions on a position corresponding to the respective
position in SbATF according to SEQ ID NO: 1. “Purity”
means the wt % retinyl acetate based on total retinoids produced
by the host organism. For more details, see text or Table 1.

| Plasmid | retAc [%] | Purity [%] |
|---|---|---|
| MB10362 | 100 | 10 |
| MB10363 | 269 | 26.9 |

With the introduction of a double mutant (see Table 1) the percentage of retinyl acetate based on total retinoids as compared to a process using an enzyme according to SEQ ID NO: 1 could be increased to more than 26 wt %.

TABLE 3B
acetylation of retinol into retinyl acetate (“retAc”) as enhanced
by action of modified ATFs comprising 3 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10364 | 475 | 47.5 |
| MB10366 | 290 | 29.0 |
| MB10369 | 375 | 37.5 |
| MB10381 | 161 | 16.1 |
| MB10387 | 655 | 65.5 |

With the introduction of a double mutant (see Table 1) the percentage of retinyl acetate based on total retinoids as compared to a process using an enzyme according to SEQ ID NO: 1 could be increased to up to 66 wt %.

TABLE 3C
acetylation of retinol into retinyl acetate (“retAc”) as enhanced
by action of modified ATFs comprising 4 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10365 | 539 | 53.9 |
| MB10367 | 473 | 47.3 |
| MB10370 | 374 | 37.4 |
| MB10372 | 237 | 23.7 |
| MB10382 | 337 | 33.7 |
| MB10384 | 561 | 56.1 |
| MB10388 | 774 | 77.4 |
| MB10390 | 577 | 57.7 |

With the introduction of 4 mutations (see Table 1) the percentage of retinyl acetate based on total retinoids as compared to a process using an enzyme according to SEQ ID NO: 1 could be improved to up to 77 wt %.

TABLE 3D
acetylation of retinol into retinyl acetate (“retAc”) as enhanced
by action of modified ATFs comprising 5 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10368 | 399 | 39.9 |
| MB10373 | 316 | 31.6 |
| MB10383 | 591 | 59.1 |
| MB10385 | 776 | 72.6 |
| MB10389 | 860 | 86.0 |
| MB10391 | 784 | 78.4 |

With the introduction of 4 mutations (see Table 1) the percentage of retinyl acetate based on total retinoids as compared to a process using an enzyme according to SEQ ID NO: 1 could be improved to up to 86 wt %.

TABLE 3E
acetylation of retinol into retinyl acetate (“retAc”) as enhanced
by action of modified ATFs comprising 6 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10374 | 237 | 23.7 |
| MB10386 | 664 | 66.4 |
| MB10392 | 798 | 79.8 |

With the introduction of 6 mutations (see Table 1) the percentage of retinyl acetate based on total retinoids as compared to a process using an enzyme according to SEQ ID NO: 1 could be improved to up to 80 wt %.

TABLE 3F
acetylation of retinol into retinyl acetate (“retAc”) as enhanced
by action of modified ATFs comprising 8 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10393 | 802 | 80.2 |

With the introduction of 4 mutations (see Table 1) the percentage of retinyl acetate based on total retinoids as compared to a process using an enzyme according to SEQ ID NO: 1 could be improved to up to 80 wt %.

TABLE 3G
acetylation of retinol into retinyl acetate (“retAc”) as enhanced
by action of modified ATFs comprising 9 amino acid substitutions
on a position corresponding to the respective position in SbATF
according to SEQ ID NO: 1. For more details, see text or Table 1.

| Plasmid | retAc [%] | Purity [%] |
|---|---|---|
| MB10362 | 100 | 10 |
| MB10394 | 112 | 11.2 |

With the introduction of 4 mutations (see Table 1) the percentage of retinyl acetate based on total retinoids as compared to a process using an enzyme according to SEQ ID NO: 1 could be improved to up to 11 wt %.

1-14. (canceled)

15. A method for modification of an enzyme catalyzing the acetylation of retinoids, particularly retinol into retinyl acetate, preferably a fungal enzyme, comprising introduction of at least 2 amino acid substitutions at positions selected from amino acid residue 34 and 434 in the polypeptide according to SEQ ID NO:1, wherein upon introduction of said at least two amino acid substitutions the activity towards formation of retinyl acetate is increased by at least about 10% compared to the activity using the respective non-modified enzyme according to SEQ ID NO: 1, and wherein the amino acid substitutions on positions corresponding to amino acid residue 34 and 434 in SEQ ID NO: 1 are selected from L34F and M434V.

16. Method according to claim 15 further comprising introduction of at least 1 amino acid substitution(s) at one or more position(s) selected from the group consisting of amino acid residue 35, 346, 371, 373, 419, 437, 478, and combinations thereof in the polypeptide according to SEQ ID NO:1, wherein upon introduction of the one or more amino acid substitution(s) the activity towards formation of retinyl acetate is increased by at least about 10%, such as in the range of 10 to 760% compared to the activity using the respective non-modified enzyme according to SEQ ID NO:1.

17. Method according to claim 15, wherein the amino acid residue to be substituted on position corresponding to position 35 in SEQ ID NO:1 is not tyrosine, and/or wherein the amino acid residue to be substituted on position corresponding to position 346 or 373 in SEQ ID NO:1 is not phenylalanine, and/or wherein the amino acid residue(s) to be substituted on position corresponding to position 371 in SEQ ID NO:1 is not valine, and/or wherein the amino acid residue(s) to be substituted on position corresponding to position 419 in SEQ ID NO:1 is not asparagine, and/or wherein the amino acid residue(s) to be substituted on position corresponding to position 437 in SEQ ID NO:1 is not arginine, and/or wherein the amino acid residue(s) to be substituted on position corresponding to position 478 in SEQ ID NO:1 is not tryptophan.

18. Method according to claim 15, wherein the amino acid residue to be introduced on position corresponding to position 35 or 478 in SEQ ID NO:1 is phenylalanine, and/or wherein the amino acid residue to be introduced on position corresponding to position 346 or 419 in SEQ ID NO: 1 is leucine, and/or wherein the amino acid residue(s) to be introduced on position corresponding to position 371 in SEQ ID NO:1 is isoleucine, and/or wherein the amino acid residue(s) to be introduced on position corresponding to position 373 in SEQ ID NO:1 is alanine, and/or wherein the amino acid residue(s) to be introduced on position corresponding to position 437 in SEQ ID NO:1 is valine.

19. Method according to claim 15, wherein an enzyme with at least 20% identity to SEQ ID NO:1 is modified via introduction of one or more amino acid substitution(s) on position corresponding to amino acid residue(s) Y35, F346, V371, F373, N419, R437, W478, and/or combinations thereof, preferably wherein the modified enzyme comprises one or more amino acid substitution(s) selected from the group consisting of Y35F, V371I, N419L, F346L, R437V, W478F, F373A, and combinations thereof, combined with amino acid substitutions L34F and M434V.

20. A process for production of retinol acetylating enzymes, preferably acetyl transferase of class [EC 2.3.1.84], having increased activity towards acetylation of retinol into retinyl acetate, said process comprising:
(a) providing a fungal enzyme involved in acetylation of retinol, particularly ATF originated from Saccharomyces, wherein said non-modified enzyme expressed in a suitable host cell and under suitable culture conditions is capable of producing up to 10 wt % retinyl acetate from retinol based on total retinoids produced by said host cell;
(b) introduction of at least 2 amino acid substitutions on positions corresponding to L34F and M434V in the polypeptide according to SEQ ID NO:1, furthermore introduction of at least 1 to 7 amino acid substitution(s) into the enzyme of (a) to generate a modified enzyme involved in acetylation of retinol, wherein the ability to convert retinol into retinyl acetate is increased by at least about 10%, such as e.g. by at least 10 to 760% when compared to the acetylation of the respective non-modified enzyme of step (a);
wherein the at least 1 or 7 amino acid substitution(s) are being located at position(s) corresponding to amino acid residue(s) being selected from the group consisting of position 35, 346, 371, 373, 419, 437, 478 in SEQ ID NO:1 and wherein the substitute amino acid(s) is/are different from Y35, F346, V371, F373, N419, R437, W478 and/or combinations thereof, wherein the amino acid residue to be introduced at position corresponding to position 35, or 478 in SEQ ID NO:1 is preferably phenylalanine, and/or wherein the amino acid residue to be introduced at position corresponding to position 346 or419 in SEQ ID NO: 1 is preferably leucine, and/or wherein the amino acid residue to be introduced at position corresponding to position 371 in SEQ ID NO:1 is preferably isoleucine, and/or wherein the amino acid residue to be introduced at position corresponding to position 373 in SEQ ID NO: 1 is preferably alanine, and/or wherein the amino acid residue to be introduced at position corresponding to position 437 in SEQ ID NO:1 is preferably valine.

21. The process according to claim 20, wherein the acetyl transferase of step (a) is selected from yeast ATF with at least about 40% identity to an enzyme according to SEQ ID NO: 1.

22. A modified enzyme involved in acetylation of retinol into retinyl acetate,
particularly fungal enzyme comprising at least 2 modifications, such as amino acid substitutions L34F and M434V being located at positions corresponding to amino acid residues 34 and 434 in the polypeptide according to SEQ ID NO:1, further comprising at least 1, 2, 3, 4, 5, 6, 7 amino acid substitution(s), said amino acid substitution(s) being located at position(s) corresponding to amino acid residue(s) being selected from the group consisting of position 35, 346, 371, 373, 419, 437, 478 and combinations thereof, in the polypeptide according to SEQ ID NO:1 or a polypeptide with a sequence of at least about 20%, such as 25, 30, 35, 40, 45, 50, 55, 60, 65, 70, 75, 80, 85, 90, 92, 95, 97, 98, 99% or up to 100% identity to SEQ ID NO:1, and wherein the substitute amino acid residue(s) are different from amino acids corresponding to Y35, F346, V371, F373, N419, R437, W478 and combinations thereof in the enzyme according to SEQ ID NO: 1, preferably wherein the amino acid residue(s) to be substituted are corresponding to residues selected from the group consisting of Y35, F346, V371, F373, N419, R437, W478 and combinations thereof in a polypeptide according to SEQ ID NO:1, particularly wherein the substitute amino acid residue to be introduced at position corresponding to position 35, or 478 in SEQ ID NO:1 is preferably phenylalanine, wherein the substitute amino acid residue to be introduced at position corresponding to position 346 or419 in SEQ ID NO: 1 is preferably leucine, wherein the substitute amino acid residue to be introduced at position corresponding to position 371 in SEQ ID NO:1 is preferably isoleucine, wherein the substitute amino acid residue to be introduced at position corresponding to position 373 in SEQ ID NO: 1 is preferably alanine, and/or wherein the substitute amino acid residue to be introduced at position corresponding to position 437 in SEQ ID NO:1 is preferably valine.

23. The modified enzyme according to claim 22 selected from a polypeptide with at least about 20%, such as 25, 30, 35, 40, 45, 50, 55, 60, 65, 70, 75, 80, 85, 90, 92, 95, 97, 98, 99% or up to 100% identity to SEQ ID NO:1, comprising one or more amino acid substitution(s) on position(s) selected from 34, 35, 346, 371, 373, 419, 434, 437 and/or 478 in SEQ ID NO:1 being different from L34, Y35, F346, V371, F373, N419, M434, R437 or W478, preferably wherein the one or more amino acid substitution(s) are selected from the group consisting of L34F, Y35F, V371I, M434V, N419L, F346L, R437V, W478F, F373A, and combinations thereof.

24. The modified enzyme according to claim 22, wherein the enzymatic activity towards formation of retinyl acetate is increased by at least about 10% compared to the respective non-modified ATF, e.g. ATF according to SEQ ID NO:1, particularly with an increase in the percentage of retinyl acetate based on total retinoids to about 86 wt %.

25. The modified enzyme according to claim 22, comprising amino acid substitution corresponding to L34F_M434V in the polypeptide according to SEQ ID NO:1.

26. A process for production of retinyl acetate, comprising the step of acetylation of retinol via action of an enzyme according to claim 22.

27. The process according to claim 26, wherein a suitable retinol producing host cell, preferably selected from Yarrowia, Saccharomyces or Escherichia coli, is transformed with and expressing an enzyme.

28. The process according to claim 26, wherein the percentage of retinyl acetate produced under suitable culture conditions is about 86 wt % based on total retinoids produced by said host cell.
